(19) United States
(12) Patent Application Publication
(10) Pub. No.: US 2017/0252038 A1
(43) Pub. Date: Sep. 7, 2017

(54) ARTHROSCOPIC MENISCAL TEAR REPAIR DEVICE

(71) Applicant: Smith & Nephew, Inc., Memphis, TN (US)

(72) Inventors: David Callaghan, Waltham, MA (US); Bernard Joseph Bourque, Rehoboth, MA (US)

(73) Assignee: Smith & Nephew, Inc., Memphis, TN (US)

(21) Appl. No.: 15/514,082

(22) PCT Filed: Oct. 13, 2015
(86) PCT No.: PCT/US2015/055241

§ 371 Date: Mar. 24, 2017

Related U.S. Application Data
(60) Provisional application No. 62/063,998, filed on Oct. 15, 2014.

(51) Int. Cl. A61B 17/06

(52) U.S. Cl. CPC A61B 17/06066; A61B 2017/00867

(57) ABSTRACT

Apparatus and methods for arthroscopic meniscal tear repair. An apparatus includes a rigid outer sheath, and a needle residing within the rigid outer sheath, the needle including a superelastic material having an angled tip portion and a lumen having a longitudinal slot, a distal end of the needle having a bend, a deflection angle of the bend dependent upon an extent to which the needle is advanced from the stiff outer sheath.

### BACKGROUND OF THE INVENTION

The present invention relates generally to arthroscopic surgery, and more particularly to an arthroscopic meniscal tear repair device.

In general, a meniscus tear is a common injury to the cartilage that stabilizes and cushions the knee joint. Surgical repair may be done by open surgery, in which a small incision is made and the knee is opened up so that a surgeon can see inside the knee and the meniscus can be repaired. Increasingly, surgeons use arthroscopic surgery to repair the meniscus. The surgeon inserts a thin tube (arthroscope) containing a camera and a light through small incisions near the knee and is able to see inside the knee without making a large incision. Surgical instruments can be inserted through other small incisions. The surgeon repairs the meniscus using sutures (stitches) or anchors.

### SUMMARY OF THE INVENTION

The following presents a simplified summary of the innovation in order to provide a basic understanding of some aspects of the invention. This summary is not an extensive overview of the invention. It is intended to neither identity key or critical elements of the invention nor delineate the scope of the invention. Its sole purpose is to present some concepts of the invention in a simplified form as a prelude to the more detailed description that is presented later.

The present invention is directed towards an arthroscopic meniscal tear repair device.

In an aspect, the invention features an apparatus including a rigid outer sheath, and a needle residing within the rigid outer sheath, the needle including a superelastic material having an angled tip portion and a lumen having a longitudinal slot, a distal end of the needle having a bend, a deflection angle of the bend dependent upon an extent to which the needle is advanced from the stiff outer sheath.

One or more of the following features may also be included. The rigid outer sheath can include a hypodermic tubing.

The rigid outer sheath can include a stainless steel tube having laser cuts at a desired curved region and the needle may include a pull wire to achieve a desired bend of the needle when advanced from the rigid outer sheath.

The needle can include a distal implant residing in the distal end of the needle, a proximal implant residing in a proximal end of the needle, and an actuator releasably linked to the proximal implant. The distal implant and the proximal implant may be tied together.

The actuator can include a body, and an arm spring loaded to the body, a distal end of the arm including a projection configured to mate with a corresponding groove in the proximal implant and lock the proximal implant when the needle is in the rigid outer sheath and release the proximal implant when the actuator is advanced from the rigid outer sheath, exposing the proximal implant.

In another aspect, the invention features a method including advancing a tissue repair device into the portion of the tissue so that a distal end of the device is disposed beyond the tear, the device including a rigid outer sheath and a needle residing within the rigid outer sheath, the needle including a superelastic material having an angled tip portion and a lumen with a longitudinal slot, a distal end of the needle having a bend, a deflection angle of the bend dependent upon an extent to which the needle is advanced from the stiff outer sheath, advancing the needle beyond a tip of the outer sheath, causing the needle to bend due to inherent properties of the needle, and advancing distally an actuator linked by a spring loaded arm to an implant within the needle beyond a tip of the needle, the spring loaded arm of the actuator releasing the implant.

One or more of the following features may also be included. The rigid outer sheath can include a hypodermic tubing.

The rigid outer sheath can include a stainless steel tube having laser cuts at a desired curved region and the needle may include a pull wire to achieve a desired bend of the needle when advanced from the rigid outer sheath.

The needle can include a distal implant residing in the distal end of the needle, a proximal implant residing in a proximal end of the needle, and an actuator releasably linked to the proximal implant. The distal implant and the proximal implant may be tied together.

The actuator can include a body, and an arm spring loaded to the body, a distal end of the arm including a projection configured to mate with a corresponding groove in the proximal implant and lock the proximal implant when the needle is in the rigid outer sheath and release the proximal implant when the actuator is advanced from the rigid outer sheath, exposing the proximal implant.

Embodiments of the invention may include one or more of the following advantages.

The arthroscopic meniscal tear repair device of the present invention device is capable of being articulated to repair/engage tears of ail segments of the meniscus.

The arthroscopic meniscal tear repair device of the present invention includes a needle having a groove and an implant having a complimentary groove wherein an actuator engages the implant when inside the needle and disengages the implant when the actuator has been advanced beyond the needle groove.

A curve of a needle can be altered depending on the distance advanced from the outer sheath due to the inherent properties of the needle.

These and other features and advantages will be apparent from a reading of the following detailed description and a review of the associated drawings. it is to be understood that both the foregoing general description and the following detailed description are explanatory only and are not restrictive of aspects as claimed.

### BRIEF DESCRIPTION OF THE DRAWINGS

The invention will be more fully understood by reference to the detailed description, in conjunction with the following figures, wherein:

FIG. 1 is an illustration of an exemplary arthroscopic meniscal tear repair device.

FIG. 2 is an illustration of a needle extended from an outer sheath.

FIG. 3 is an illustration of a needle of the exemplary arthroscopic meniscal tear repair device.

FIG. 4 is an illustration of pre-tied proximal and distal implants.

FIG. 5 is a diagram of an actuator in an unlocked position.

FIG. 6 is a diagram of the actuator in a locked position.

FIG. 7 is a flow diagram.

### DETAILED DESCRIPTION

The subject innovation is now described with reference to the drawings, wherein like reference numerals are used to refer to like elements throughout. In the following description, for purposes of explanation, numerous specific details are set forth in order to provide a thorough understanding of the present invention. It may be evident, however, that the present invention may be practiced without these specific details. In other instances, well-known structures and devices are shown in block diagram form in order to facilitate describing the present invention.

As used in this application, the term “or” is intended to mean an inclusive “or” rather than an exclusive “or.” That is, unless specified otherwise, or clear from context, “X employs A or B” is intended to mean any of the natural inclusive permutations. That is, if X employs A, X employs B, or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form.

The present description will be used with reference to arthroscopic meniscal tear repair, but it should be understood that the technology and methods of the present invention may have other applications for reconstruction and repair of other parts of body, such as, for example, hip capsule closure and repair.

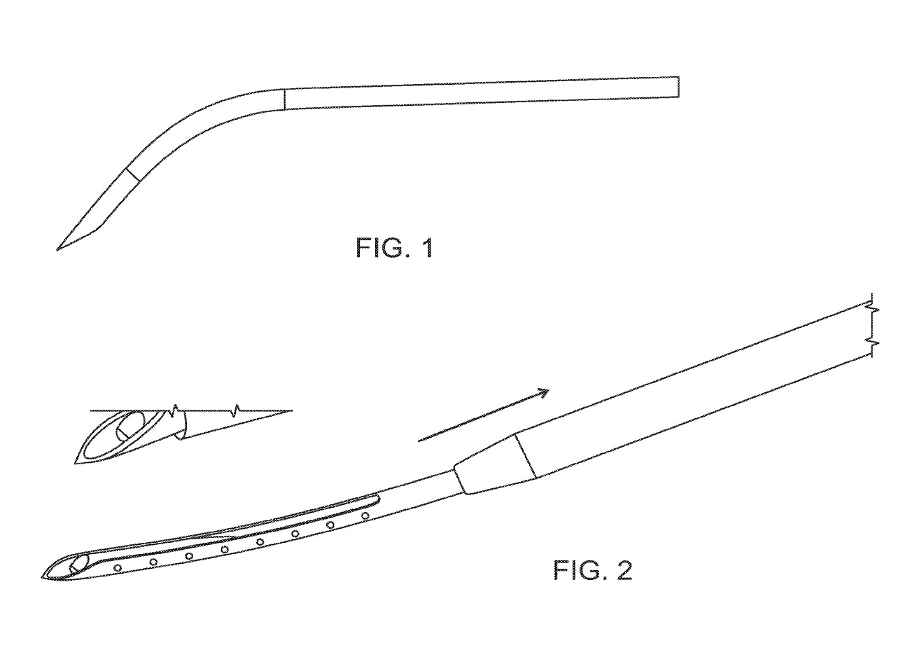

As shown in FIG. 1, an exemplary arthroscopic meniscal tear repair device 10 includes an outer sheath 15 and a needle 20. The outer sheath 15 is a relatively rigid thick-walled stainless hypodermic tubing. In alternate embodiments, other materials may be used to form the outer sheath.

As shown in FIG. 2, the outer sheath 15 is designed to maintain a straight configuration throughout an entire surgical procedure, while the inherent properties of the needle 20, fully described below, cause it to bend as it is distally advanced from the outer sheath 15.

Referring again to FIG. 1, in the illustrated embodiment, the needle 20 is formed from a superelastic material, such as nickel titanium. In a preferred embodiment, the needle is formed from 17 gage (0.057) Nitinol. A distal end 25 of the needle 20 is manufactured with a bend. The inherent superelastic property of the nickel titanium needle 20 enables it to deform from its natural bended configuration to a straight configuration without yielding white recessed within the outer sheath 15. As the distal end 25 of the needle 20 is advanced from the outer sheath 15, the needle 20 bends. The extent to which the needle 20 is advanced from the outer sheath 15 determines the extent of the deflection angle of the needle 20. When the needle 20 is retracted into the outer sheath 16, the bend in the needle 20 straightens due to the physical constraints imposed by the rigid outer sheath 15. A curved needle 20 that has been advanced distally from the outer sheath 15 is illustrated in FIG. 1.

In one alternate embodiment, the arthroscopic meniscal tear repair device is manufactured using a stainless tube that has laser cuts at the desired curved region. In addition, there may be a pull wire to achieve the desired curvature in the needle.

In another alternate embodiment, the arthroscopic meniscal tear repair device uses a coil at the curved region to enable the device to curve upon applying force to the pull wire.

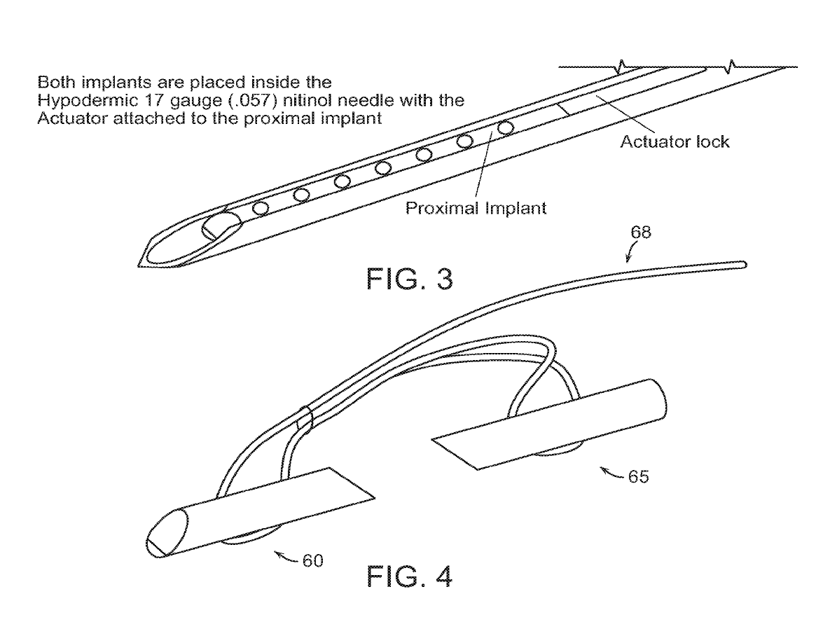

As shown in FIG. 3, the needle 20 includes an angled tip portion 50 and a lumen 55 having a longitudinal slot 57. The lumen 55 is exposed at the tip portion 50. The lumen 55 includes a distal implant 60, a proximal implant 65 and an actuator 70.

As shown in FIG. 4, the distal implant 60 and proximal implant 65 may be pre-tied by one or more sutures 68, such as with the sliding knot feature of the FAST-FIX™ 360 Meniscal Repair System manufactured by Smith & Nephew, Inc., of Andover, Mass. US. The longitudinal slot 57 of the lumen 55 (shown in FIG. 3) enables access to and/or manipulation of the sutures and/or sliding knot. Pre-tying the distal implant 60 and proximal implant 55 enable a release of the distal implant 60 during distal advancement of the actuator 70 from the needle 20 and prevents the proximal implant 65 from failing from the needle 20 prematurely as the actuator 70 is distally advanced from the needle 20.

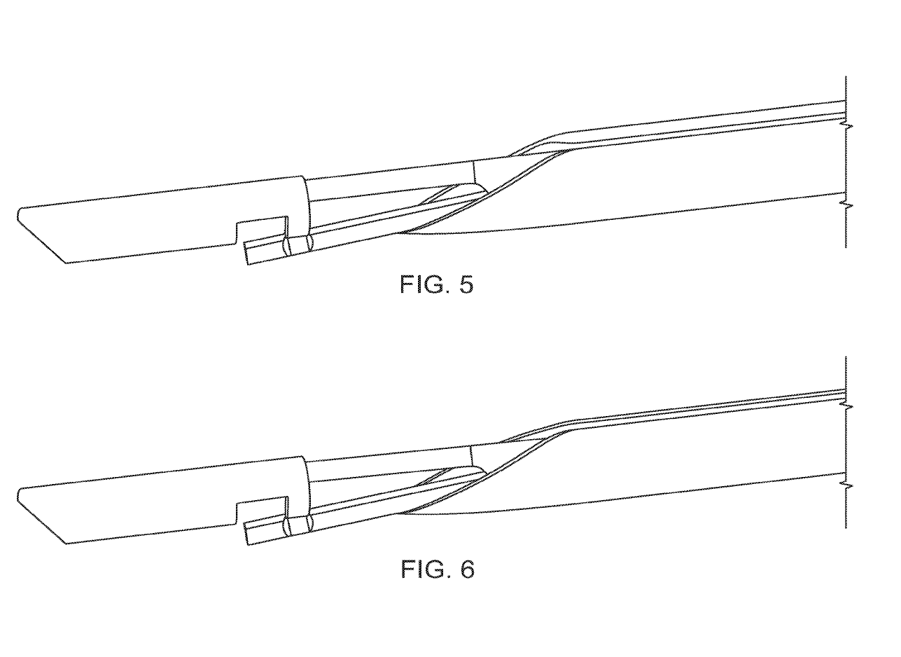

As shown in FIG. 5, the actuator 70 includes a body 75 and a spring loaded arm 80. The spring loaded arm 80 includes a projection 85 located at a distal end of the arm 80. The projection 85 is configured to mate with a corresponding groove 90 in the proximal implant 65. When the proximal implant 65 is placed within the needle 20 and loaded proximally into the outer sheath 15, the spring loaded arm 80 compresses, causing the projection 85 to lock into the groove 90 of the proximal implant 65. When actuator 70 is advanced distally from the needle 20, the proximal implant 65 is exposed from the outer sheath 15, the spring loaded arm 80 unlocks from the groove 90, releasing the proximal implant 65. More specifically, as the actuator 70 is advanced distally from the needle 20, arm 80 of the actuator 70 springs open and disengages from the groove 90 to release the proximal implant 65 from the needle 20. In FIG. 3, the proximal implant 65 and the arm 80 are shown advanced beyond the angled tip portion 50 of the needle 20.

As shown in FIG. 6, the proximal implant 65 and actuator 70 are shown within the lumen 55 of the needle 20. In such a configuration, the arm 80 is locked into the groove 90 of the proximal implant 65, preventing the proximal implant 65 from being prematurely disengaged from the needle 20 until the actuator 70 is advanced from the needle 20.

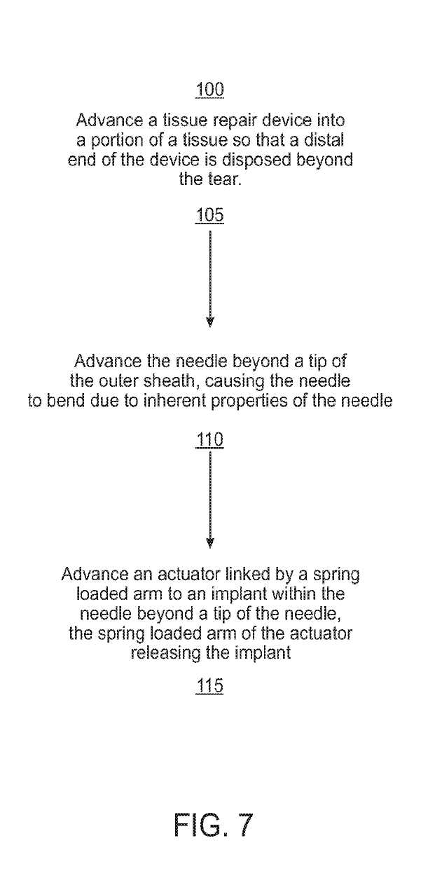

As shown in FIG. 7, a process 100 for repairing a tear in a portion of tissue includes advancing (105) a tissue repair device into the portion of the tissue so that a distal end of the device is disposed beyond the tear. The device includes a rigid outer sheath and a needle residing within the rigid outer sheath, the needle including a superelastic material having an angled tip portion and a lumen, a distal end of the needle having a bend, a deflection angle of the bend adjustable on a distance the needle is advanced from the stiff outer sheath.

Process 100 advances (110) the needle beyond a tip of the outer sheath, causing the needle to bend due to the inherent properties of the needle.

Process 100 advances (115) distally an actuator linked by a spring loaded arm to an implant within the needle beyond a tip of the needle, the spring loaded arm of the actuator releasing the implant.

Although the above-described device was in the context of arthroscopic meniscal tear repair, it should be understood that the technology and methods of the present invention may have other applications for reconstruction and repair of other parts of body, such as, for example, hip capsule closure and repair. The illustrated and described embodiments should not be considered to be limiting in any way.

The fixation members, limiting elements, and flexible members of the above embodiments can be formed of a biodegradable material.

The devices and techniques described above can be applied to other anatomical regions to reattach tissue to bone or repair a tear in soft tissue, such as the biceps tendons, the lateral collateral ligament, the medial collateral ligament, the popliteal ligament, and the hip.

While this invention has been particularly shown and described with references to preferred embodiments thereof, it will be understood by those skilled in the art that various changes in form and details may be made therein without departing from the spirit and scope of the present application as defined by the appended claims. Such variations are intended to be covered by the scope of this present application. As such, the foregoing description of embodiments of the present application is not intended to be limiting. Rather, any limitations to the invention are presented in the following claims.

What is claimed is:

1. An apparatus comprising:
a rigid outer sheath; and
a needle residing within the rigid outer sheath, the needle comprising a superelastic material having an angled tip portion and a lumen having a longitudinal slot, a distal end of the needle having a bend, a deflection angle of the bend dependent upon an extent to which the needle is advanced from the stiff outer sheath.

2. The apparatus of claim 1 wherein the rigid outer sheath comprises a hypodermic tubing.

3. The apparatus of claim 1 wherein the rigid outer sheath comprises a stainless steel tube having laser cuts at a desired curved region.

4. The apparatus of claim 3 further comprising a pull wire to achieve a desired bend of the needle when advanced from the rigid outer sheath.

5. The apparatus of claim 1 wherein the needle comprises:
a distal implant residing in the distal end of the needle;
a proximal implant residing in a proximal end of the needle; and
am actuator releasably linked to the proximal implant.

6. The apparatus of claim 5 wherein the distal implant and the proximal implant are tied together.

7. The apparatus of claim 6 wherein the actuator comprises:
a body; and
an arm spring loaded to the body, a distal end of the arm comprising a projection configured to mate with a corresponding groove in the proximal implant and look the proximal implant when the needle is in the rigid outer sheath and release the proximal implant when the actuator is advanced from the rigid outer sheath, exposing the proximal implant.

8. A method for repairing a tear in a portion of tissue, the method comprising:
advancing a tissue repair device into the portion of the tissue so that a distal end of the device is disposed beyond the tear, the device comprising a rigid outer sheath and a needle residing within the rigid outer sheath, the needle comprising a superelastic material having an angled tip portion and a lumen with a longitudinal slot, a distal end of the needle having a bend, a deflection angle of the bend dependent upon an extent to which the needle is advanced from the stiff outer sheath;
advancing the needle beyond a tip of the outer sheath, causing the needle to bend due to inherent properties of the needle; and
advancing distally an actuator linked by a spring loaded arm to an implant within the needle beyond a tip of the needle, the spring loaded arm of the actuator releasing the implant.

9. The method of claim 8 wherein the rigid outer sheath comprises a hypodermic tubing.

10. The method of claim 8 wherein the rigid outer sheath comprises a stainless steel tube having laser cuts at a desired curved region.

11. The method of claim 10 wherein the needle further comprises a pull wire to achieve a desired bend of the needle when advanced from the rigid outer sheath.

12. The method of claim 8 wherein the implant is tied to a second implant.

13. The method of claim 8 wherein the actuator comprises:
a body; and
the arm spring loaded to the body, a distal end of the arm comprising a projection configured to mate with a corresponding groove in the implant and lock the implant when the needle is in the rigid outer sheath and release the implant when the actuator is advanced from the rigid outer sheath, exposing the implant.
